(19) United States
(12) Patent Application Publication
(10) Pub. No.: US 2005/0074413 A1
(43) Pub. Date: Apr. 7, 2005

(54) Aerosol composition comprising silicate particles and surfactants

(76) Inventors: Emmanuelle Belli, Asnierres (FR); Audrey Gilles, Velizy (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/479,344

(22) PCT Filed: May 31, 2002
(86) PCT No.: PCT/FR02/01849

(30) Foreign Application Priority Data
May 31, 2001 (FR) 0107154

(51) Int. Cl. A61K 7/06; A61K 7/11; A61K 9/00

(52) U.S. Cl. 424/47; 424/70.12

(57) ABSTRACT

The invention concerns a cosmetic composition, in particular for the hair, packaged in an aerosol device containing a propellant and a liquid phase which comprises, in a cosmetically acceptable medium: (i) solid particles containing at least 10 wt. % of at least a silicate, and (ii) at least a surfactant; said solid particles containing less than 1 wt. % of aluminium.

The present invention relates to a composition packaged in an aerosol device comprising a propellant and a liquid phase, which contains, in a cosmetically acceptable medium, silicate particles, to a cosmetic process for treating the hair and to a use as a styling product.

Styling products are usually used to construct and structure the hairstyle and give it long-lasting hold. To do this, film-forming polymers are introduced into a cosmetically acceptable medium. However, certain polymers result in hardening of the hair and/or a sticky nature. As a result, the hair is often stuck together, and the hair shape is destroyed upon disentangling.

To overcome these drawbacks, it has already been proposed to use solid powder in cosmetic compositions, such as metal oxide powders. For example, patent U.S. Pat. No. 3,819,827 describes hairsetting products comprising from 0.2% to 6% by weight of aluminum oxide particles with a particle size of about 30 μm.

Similarly, patent application EP 1 005 849 describes the use of fine solid mineral powders, for instance calcium carbonate, aluminum hydroxide, magnesium carbonate, calcium phosphate, calcium oxalate or barium sulfate powders, in cosmetic hair compositions containing at least two liquid phases. It is pointed out that such compositions give the hair especially advantageous effects when the composition is packaged, without a propellant, in a device capable of spraying the composition onto the hair.

Patent DE 25 42 338 describes propellant-free lotions and sprays for fixing the hair, comprising silicate particles, in an alcoholic or aqueous-alcoholic medium, the silicate concentration being between 0.1% and 4%.

The Applicant has found, surprisingly and unexpectedly, that by selecting silicate particles as solid particles, it is possible to obtain good cosmetic properties and to obtain styling without making the hair sticky or overloading it, insofar as the composition is packaged in a particular device, namely an aerosol device, when the cosmetic composition also comprises at least one surfactant.

One subject of the invention is a cosmetic composition, especially a hair composition, packaged in an aerosol device containing a propellant and a liquid phase, which comprises, in a cosmetically acceptable medium:
(i) solid particles containing at least 10% by weight of at least one silicate, and
(ii) at least one surfactant, said solid particles containing less than 1% aluminum.

Another subject of the present invention consists of a cosmetic hair treatment process using the composition according to the invention.

Another subject of the invention is the use of the cosmetic hair composition, especially for fixing and/or holding the hair in a desired shape.

The invention is also directed toward the use, in hair cosmetics, especially for fixing and/or holding the hair in a desired shape, of a cosmetic composition packaged in an aerosol device containing a propellant and a liquid phase, which comprises, in a cosmetically acceptable medium, solid particles containing at least 10% by weight of at least one silicate, said solid particles containing less than 1% by weight of aluminum.

Preferably, the solid particles contain between 0 and 1% aluminum and more preferably between 0 and 0.5% aluminum.

Other subjects, characteristics, aspects and advantages of the invention will emerge even more clearly on reading the description and the various examples that follow.

Particles

Advantageously, the solid particles have a number-average primary size of between 2 nm and 1 μm.

The particles containing at least 10% by weight of at least one silicate preferably have a number-average primary size of between 5 and 500 nm and more preferably between 10 and 250 nm.

The particles according to the invention may be in any form, for example in the form of spheres, flakes, needles, platelets or totally random forms.

The particles are preferably sodium, magnesium and/or lithium silicates, for instance the compounds sold by the company Laporte under the names Laponite XLG and Laponite XLS.

For the purposes of the present invention, the expression “primary particle size” means the maximum size that it is possible to measure between two diametrically opposite points on an individual particle. The size may be determined, for example, by transmission electron microscopy or by measuring the specific surface area by the BET method, or alternatively using a laser granulometer.

When the particles are formed from silicate and other fillers, the silicate is in free form and does not form chemical bonds with the other fillers. This is then a case of an alloy between the silicate and other fillers, especially with metal or metalloid oxides, in particular obtained by thermal fusion of these various constituents.

When the particles containing at least 10% by weight of at least one silicate also comprise a metal or metalloid oxide, this oxide is chosen especially from silicon oxide, boron oxide and aluminum oxide.

Preferably, the particles contain at least 50% by weight of silicate and better still at least 70% by weight of silicate, and particles consisting of more than 90% by weight of silicate are particularly preferred according to the present invention.

The silicate that is suitable for use in the compositions of the present invention may be of natural origin or may be of synthetic origin.

The silicate-containing particles according to the invention are especially used in an amount of between 0.01% and 10% by weight and preferably between 0.5% and 3% by weight relative to the total weight of the composition.

The composition according to the invention may also contain other types of particle, for example titanium oxide, zinc oxide or aluminum oxide particles.

Surfactant

The surfactants that are suitable for carrying out the present invention are especially the following:

(i) Anionic Surfactant(s):

In the context of the present invention, their nature is not a truly critical feature.

Thus, as examples of anionic surfactants that can be used, alone or as mixtures, in the context of the present invention, mention may be made in particular (nonlimiting list) of salts (in particular alkaline salts, especially sodium salts, ammonium salts, amine salts, amino alcohol salts or alkaline-earth metal salts (of magnesium) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, the alkyl or acyl radical of all of these various compounds preferably containing from 12 to 20 carbon atoms, and the aryl radical preferably denoting a phenyl or benzyl group. Among the anionic surfactants which can also be used, mention may also be made of fatty acid salts such as the salts of oleic, ricinoleic, palmitic and stearic acids, coconut oil acid or hydrogenated coconut oil acid; acyl lactylates in which the acyl radical contains 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, in particular those containing from 2 to 50 ethylene oxide groups, and mixtures thereof.

Among the anionic surfactants, it is preferred according to the invention to use alkyl sulfate salts and alkyl ether sulfate salts and mixtures thereof.

(ii) Nonionic surfactant(s):

The nonionic surfactants are, themselves also, compounds that are well known per se (see in particular in this respect “Handbook of Surfactants” by M. R. Porter, published by Blackie & Son (Glasgow and London), 1991, pp. 116-178) and, in the context of the present invention, their nature is not a critical feature. Thus, they can be chosen in particular from (nonlimiting list) polyethoxylated, polypropoxylated or polyglycerolated fatty acids, alkylphenols, α-diols or alcohols having a fatty chain containing, for example, 8 to 18 carbon atoms, it being possible for the number of ethylene oxide or propylene oxide groups to range in particular from 2 to 50 and for the number of glycerol groups to range in particular from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄) alkylamine oxides or N-acylaminopropylmorpholine oxides.

(iii) Amphoteric Surfactant(s):

The amphoteric surfactants, whose nature is not a critical feature in the context of the present invention, can be, in particular (nonlimiting list), aliphatic secondary or tertiary amine derivatives in which the aliphatic radical is a linear or branched chain containing 8 to 22 carbon atoms and containing at least one water-soluble anionic group (for example carboxylate, sulfonate, sulfate, phosphate or phosphonate); mention may also be made of (C₈-C₂₀)alkylbetaines, sulfobetaines, (C₈-C₂₀)alkylamido(C₁-C₆)alkylbetaines or (C₈-C₂₀)alkylamido(C₁-C₆)alkylsulfobetaines.

Among the amine derivatives, mention may be made of the products sold under the name Miranol®, as described in U.S. Pat. Nos. 2,528,378 and 2 781 354 and having the structures:
R₂—CONHCH₂CH₂—N(R₃) (R₄) (CH₂COO—) (2)
in which: R₂ denotes an alkyl radical derived from an acid R₂—COOH present in hydrolyzed coconut oil, a heptyl, nonyl or undecyl radical, R₃ denotes a β-hydroxyethyl group and R₄ denotes a carboxymethyl group; and
   R₂′-CONHCH₂CH₂—N(B)(C) (3)
in which:

B represents —CH₂CH₂OX′, C represents —(CH₂)_{z}—Y′, with z=1 or 2,
X′ denotes the —CH₂CH₂—COOH group or a hydrogen atom,
Y′ denotes —COOH or the —CH₂—CHOH—SO₃H radical,
R₂, denotes an alkyl radical of an acid R₉-COOH present in coconut oil or in hydrolyzed linseed oil, an alkyl radical, in particular a C₇, Cg, C₁₁ or C₁₋₃ alkyl radical, a C₁₋₇ alkyl radical and its iso form, an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloampho-diacetate, disodium cocoamphodipropionate, disodium lauroamphodipropionate, disodium caprylampho-dipropionate, disodium capryloamphodipropionate, lauroamphodipropionic acid, cocoamphodipropionic acid.

By way of example, mention may be made of the cocoamphodiacetate sold under the trade name Miranol®C2M concentrate by the company Rhodia.

Mixtures of surfactants and in particular mixtures of anionic surfactants and of amphoteric or nonionic surfactants are preferably used in the compositions in accordance with the invention. A mixture that is particularly preferred is a mixture consisting of at least one anionic or nonionic surfactant and of at least one amphoteric surfactant.

It is preferred to use an anionic surfactant chosen from sodium, triethanolamine or ammonium (C₁₂-C₁₄)alkyl sulfates, sodium (C₁₂-C₁₋₄)alkyl ether sulfates oxyethylenated with 2.2 mol of ethylene oxide, sodium cocoyl isethionate and sodium (C₁₄—C₁₆)-α-olefin sulfonate, and mixtures thereof with:
either an amphoteric surfactant such as the amine derivatives known as disodium cocoampho-dipropionate or sodium cocoamphopropionate sold especially by the company Rhodia under the trade name “Miranol® C2M CONC” as an aqueous solution containing 38% active material, or under the name Miranol® C32;
or an amphoteric surfactant of zwitterionic type, such as alkylbetaines, in particular the cocoylbetaine sold under the name “Dehyton AB 30” as an aqueous solution containing 32% AM by the company Henkel, or alkylamidobetaines such as Tegobetaine F50 sold by the company Goldschmidt.

Cationic Surfactants

The composition according to the invention comprises one or more cationic surfactants that are well known per se, such as optionally polyoxyalkylen-ated primary, secondary or tertiary fatty amine salts and quaternary ammonium salts, and mixtures thereof.

Examples of quaternary ammonium salts that may especially be mentioned include:
those having the general formula (VI) below: 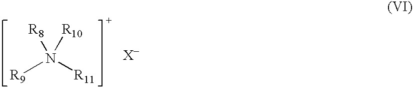
in which the radicals R₈ to R₁₁, which may be identical or different, represent a linear or branched aliphatic radical containing from 1 to 30 carbon atoms or an aromatic radical such as aryl or alkylaryl. The aliphatic radicals may comprise hetero atoms especially such as oxygen, nitrogen, sulfur and halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (Cl₂—C₂₋₂)alkyl acetate and hydroxyalkyl radicals, containing from about 1 to 30 carbon atoms; X⁻ is an anion chosen from the group of halides, phosphates, acetates, lactates, (C₂-C₆)alkyl sulfates, alkyl sulfonates and alkylaryl sulfonates;
quaternary ammonium salts of imidazoline, for instance those of formula (VII) below: 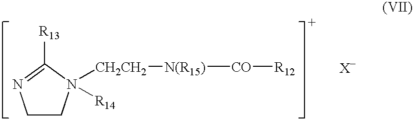
in which R₁₂ represents an alkenyl or alkyl radical containing from 8 to 30 carbon atoms, for example fatty acid derivatives of tallow, R₁₃ represents a hydrogen atom, a C₁-C₄ alkyl radical or an alkenyl or alkyl radical containing from 8 to 30 carbon atoms, R₁₄ represents a C₁-C₄ alkyl radical, R₁₅ represents a hydrogen atom or a C₁-C₄ alkyl radical, and X⁻ is an anion chosen from the group of halides, phosphates, acetates, lactates, alkyl sulfates, alkyl sulfonates or alkylaryl sulfonates. R₁₂ and R₁₃ preferably denote a mixture of alkenyl or alkyl radicals containing from 12 to 21 carbon atoms, such as, for example, fatty acid derivatives of tallow, R₁₄ denotes a methyl radical and R₁₅ denotes a hydrogen atom. Such a product is sold, for example, under the name Rewoquat W75 by the company Rewo,
the diquaternary ammonium salts of formula (VIII): 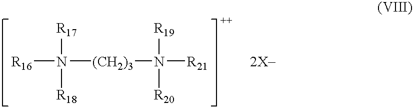
in which R₁₆ denotes an aliphatic radical containing from about 16 to 30 carbon atoms, R₁₇, R₁₈, R₁₉, R₂₀ and R₂₁, which may be identical or different, are chosen from hydrogen and an alkyl radical containing from 1 to 4 carbon atoms, and X⁻ is an anion chosen from the group of halides, acetates, phosphates, nitrates and methyl sulfates. Such diquaternary ammonium salts in particular comprise propanetallowdiammonium dichloride;
the quaternary ammonium salts containing at least one ester function, such as those of formula (IX) below: 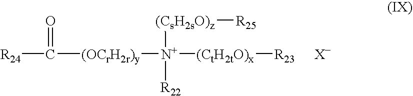
in which:
R₂₂ is chosen from C₁-C₆ alkyl radicals and C₁-C₆ hydroxyalkyl or dihydroxyalkyl radicals;
R₂₃ is chosen from:
   a radical R₂₆ 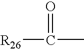
   linear or branched, saturated or unsaturated C₁-C₂₂ hydrocarbon-based radicals R₂₇,
   a hydrogen atom, R₂₅ is chosen from:
   a radical R₂₈ 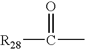
   linear or branched, saturated or unsaturated C₁-C₆ hydrocarbon-based radicals R₂₉,
   a hydrogen atom,
R₂₄, R₂₆ and R₂₈, which may be identical or different, are chosen from linear or branched, saturated or unsaturated C₇-C₂₁ hydrocarbon-based radicals;
r, s and t, which may be identical or different, are integers ranging from 2 to 6;
y is an integer ranging from 1 to 10;
x and z, which may be identical or different, are integers ranging from 0 to 10;
X⁻ is a simple or complex, organic or mineral anion;
with the proviso that the sum x+y+z is from 1 to 15, that when x is 0, then R₂₃ denotes R₂₇ and that when z is 0, then R₂₅ denotes R₂₉.

The alkyl radicals R₂₂ may be linear or branched, but more particularly linear.

R₂₂ preferably denotes a methyl, ethyl, hydroxyethyl or dihydroxypropyl radical, and more particularly a methyl or ethyl radical.

Advantageously, the sum x+y+z is from 1 to 10.

When R₂₃ is a hydrocarbon-based radical R₂₇, it may be long and may contain from 12 to 22 carbon atoms, or may be short and may contain from 1 to 3 carbon atoms.

When R₂₅ is a hydrocarbon-based radical R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁, hydrocarbon-based radicals, and more particularly from linear or branched, saturated or unsaturated C₁₁-C₂₁, alkyl and alkenyl radicals.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion is preferably a halide (chloride, bromide or iodide) or an alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function, may be used.

The anion X⁻ is even more particularly chloride or methyl sulfate.

Use is made more particularly in the composition according to the invention of the ammonium salts of formula (IX) in which:
R₂₂ denotes a methyl or ethyl radical,
x and y are equal to 1;
z is equal to 0 or 1;
r, s and t are equal to 2;
R₂₃ is chosen from:
   a radical R₂₆ 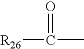
   methyl, ethyl or C₁₄-C₂₂ hydrocarbon-based radicals,
   a hydrogen atom;
R₂₅ is chosen from:
   a radical R₂₈ 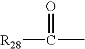
   a hydrogen atom;
R₂₄, R₂₆ and R₂₈, which may be identical or different, are chosen from linear or branched, saturated or unsaturated Cl₃—C₁₋₇ hydrocarbon-based radicals and preferably from linear or branched, saturated or unsaturated C₁₃-C₁₇ alkyl and alkenyl radicals.

The hydrocarbon-based radicals are advantageously linear.

Examples that may be mentioned include the compounds of formula (IX) such as the diacyloxyethyl-dimethylammonium, diacyloxyethylhydroxyethylmethyl-ammonium, monoacyloxyethyldihydroxyethylmethylammonium, triacyloxyethylmethylammonium and monoacyloxyethyl-hydroxyethyldimethylammonium salts (chloride or methyl sulfate in particular), and mixtures thereof. The acyl radicals preferably contain 14 to 18 carbon atoms and are obtained more particularly from a plant oil such as palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesteri-fication of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The composition according to the invention preferably contains a mixture of quaternary ammonium salts of mono-, di- and triesters with a weight majority of diester salts.

Examples of mixtures of ammonium salts that may be used include the mixture containing 15% to 30% by weight of acyloxyethyldihydroxyethylmethylammonium methyl sulfate, 45% to 60% of diacyloxyethylhydroxy-ethylmethylammonium methyl sulfate and 15% to 30% of triacyloxyethylmethylammonium methyl sulfate, the acyl radicals containing from 14 to 18 carbon atoms and being derived from palm oil that is optionally partially hydrogenated.

It is also possible to use the ammonium salts containing at least one ester function that are described in patents U.S. Pat. No. 4,874,554 and U.S. Pat. No. 4,137,180.

Among the quaternary ammonium salts of formula (VI), the ones preferably used are, on the one hand, tetraalkylammonium chlorides such as, for example, dialkyldimethylammonium chlorides or alkyltrimethylammonium chlorides, in which the alkyl radical contains from about 12 to 22 carbon atoms, in particular behenyltrimethylammonium chloride, distearyldimethylammonium chloride, cetyltrimethyl-ammonium chloride, or benzyldimethylstearylammonium chloride, or, on the other hand, palmitylamidopropyl-trimethylammonium chloride or stearamidopropyldimethyl-(myristyl acetate)ammonium chloride sold under the name Ceraphyl® 70 by the company Van Dyk.

The cationic surfactants that are particularly preferred in the composition of the invention are chosen from quaternary ammonium salts, and in particular from behenyltrimethylammonium chloride and palmitylamidopropyltrimethylammonium chloride.

The composition according to the invention preferably comprises the surfactant(s) in an amount of between 0.1% and 10% by weight, better still between 0.5% and 8% by weight and even more preferably between 1% and 5% by weight relative to the total weight of the composition.

The surfactant that is advantageously chosen is an amphoteric or nonionic surfactant.

Composition

The cosmetically acceptable medium comprises water and/or at least one cosmetically acceptable solvent chosen especially from C₁-C₄ lower alcohols, for instance ethanol, isopropanol, tert-butanol and n-butanol; polyols, for instance propylene glycol; polyol ethers; acetone, and mixtures thereof. The solvent that is particularly preferred in the invention is ethanol.

The amount of water is preferably between 10% and 95% and even more preferably between 15% and 45%.

The propellant is preferably chosen from dimethyl ether, C₃ to C₅ alkanes, for instance n-butane, propane or isobutane, halohydrocarbons, for instance 1,1-difluoroethane, mixtures of dimethyl ether and of C₃₋₅ alkanes, and mixtures of 1,1-difluoroethane and of dimethyl ether and/or of C₃₋₅ alkanes.

The propellant is more preferably chosen from dimethyl ether and C₃ to C₅ alkanes, and mixtures thereof.

The composition according to the invention may comprise common cosmetic additives chosen especially from adhesives, reducing agents, for instance thiols, fatty substances, thickeners, softeners, antifoams, screening agents, antiperspirants, acidifying agents, basifying agents, dyes, pigments, fragrances, preserving agents, anionic, cationic, nonionic or amphoteric, fixing or nonfixing polymers, volatile or nonvolatile, modified or unmodified, soluble or insoluble silicones, plant, animal, mineral or synthetic oils, fragrances, proteins, vitamins and glycerol, and mixtures thereof.

A person skilled in the art will take care to select the optional additives and the amount thereof such that they do not harm the properties of the compositions of the present invention.

These additives are especially present in the composition according to the invention in an amount ranging from 0 to 20% by weight relative to the total weight of the composition.

The propellant is especially present in an amount of between 2% and 90% by weight, preferably between 4% and 80% by weight and better still between 30% and 70% by weight relative to the total weight of the composition.

The compositions in accordance with the invention are packaged in a standard cosmetic aerosol device. The sprayed compositions may be in the form of a spray or a mousse.

The compositions in accordance with the invention, sprayed from the aerosol device, may be used in rinse-out or leave-in application, as compositions for fixing and/or holding the hair, haircare compositions, shampoos, hair conditioning compositions, such as compositions intended to give the hair softness, or hair makeup compositions.

The present invention also relates to a cosmetic hair treatment process, which consists in applying an effective amount of a composition as described above to the hair and in optionally rinsing, after an optional leave-in time.

According to one preferred embodiment of the invention, the composition, sprayed from the aerosol device, may be used as a leave-in styling product.

In particular, the compositions in accordance with the invention are used for shaping and/or holding the hair.

The examples that follow illustrate the present invention and should not be considered as limiting the invention in any way.

### EXAMPLES

Three styling products were prepared using the ingredients below, the percentages being expressed on a weight basis:

### Example 1

| | |
|---|---|
| Laponite XLG (Laporte) | 0.8% |
| Tegobetaine HS (Goldschmidt) | 0.4% |
| Brij 30 (Uniqema) | 0.2% |
| Viscophobe DB 1000 (Amerchol) | 0.86% |
| Preserving agents | qs |
| Aerogaz 3.2 (1) | 6% |
| Water | qs 100% |

(1) mixture of isobutane, propane and butane,sold by Atochem

### Example 2

| | |
|---|---|
| Laponite XLG (Laporte) | 0.5% |
| Tegobetaine HS (Goldschmidt) | 0.5% |
| Brij 30 (Uniqema) | 0.2% |
| Preserving agents | qs |
| Butane 3.2 | 6% |
| Water | qs 100% |

### Example 3

| | |
|---|---|
| Laponite XLG (Laporte) | 1% |
| Tegobetaine HS (Goldschmidt) | 0.5% |
| Brij 30 (Uniqema) | 0.2% |
| Preserving agents | qs |
| Butane 3.2 | 6% |
| Water | qs 100% |

These products are in the form of an aerosol mousse and are preferably applied to wet hair. After shaping, a good styling effect and long-lasting hold are observed, with no cosmetic drawbacks.

1. A composition, comprising a propellant and a liquid cosmetic composition, wherein the liquid cosmetic composition comprises, in a cosmetically acceptable medium:
(i) solid particles comprising at least 10% by weight of at least one silicate, and
(ii) at least one surfactant;
wherein said solid particles comprise less than 1% by weight of aluminum.

2. The composition as claimed in claim 1, wherein the silicate is at least one of a sodium, magnesium or lithium silicate.

3. The composition as claimed in claim 1, wherein the solid particles have a number-average primary size of between 2 nm and 1 μm.

4. The composition as claimed in claim 1, wherein the solid particles comprising at least 10% by weight of at least one silicate have a number-average primary size of between 5 and 500 nm.

5. The composition as claimed in claim 1, wherein the solid particles comprise at least 50% by weight of at least one silicate.

6. The composition as claimed in claim 1, wherein the silicate comprising particles are present in an amount of between 0.01% and 10% by weight relative to the total weight of the composition.

7. The composition as claimed in claim 1, wherein the surfactant is present in an amount of between 0.1% and 10% by weight, relative to the total weight of the composition.

8. The composition as claimed in claim 1, wherein the surfactant is an amphoteric or nonionic surfactant.

9. The composition as claimed in claim 1, wherein the propellant is at least one selected from the group consisting of dimethyl ether, a C₃ to C₅ alkane, n-butane, propane, isobutane, a halohydrocarbon, 1,1-difluoroethane, a mixture of dimethyl ether and a C₃₋₅ alkane, and a mixture of 1,1-difluoroethane and at least one of dimethyl ether or a C₃₋₅ alkane.

10. The composition as claimed in claim 1, wherein the propellant is at least one of dimethyl ether, a C₃ to C₅ alkane, or a mixture thereof.

11. The composition as claimed in claim 1, wherein the propellant is present in an amount of between 2% and 90% by weight, relative to the total weight of the composition.

12. The composition as claimed in claim 1, further comprising one or more cosmetic additives selected from the group consisting of an adhesive; a reducing agent; a thio; a fatty substance; a thickener; a softener; an antifoam; a screening agent; an antiperspirant; an acidifying agent; a basifying agent; a dye; a pigment; a fragrance; a preserving agent; an anionic, cationic, nonionic or amphoteric, fixing or nonfixing polymer; volatile or nonvolatile, modified or unmodified, soluble or insoluble silicone; a plant, animal, mineral or synthetic oil, fragrance; protein; vitamin; or glycerol; and mixtures thereof.

13-15. (canceled)

16. The cosmetic composition as claimed in claim 1, in the form of an aerosol.

17. The composition as claimed in claim 1, wherein the solid particles comprising at least 10% by weight of at least one silicate have a number-average primary size of between 10 and 250 nm.

18. The composition as claimed in claim 1, wherein the solid particles comprise at least 70% by weight of at least one silicate.

19. The composition as claimed in claim 1, wherein the solid particles comprise at least 90% by weight of at least one silicate.

20. The composition as claimed in claim 1, wherein the silicate-comprising particles are present in an amount of between 0.5% and 3% by weight relative to the total weight of the composition.

21. The composition as claimed in claim 1, wherein the surfactant is present in an amount of between 0.5% and 8% by weight relative to the total weight of the composition.

22. The composition as claimed in claim 1, wherein the surfactant is present in an amount of between 1% and 5% by weight relative to the total weight of the composition.

23. The composition as claimed in claim 1, wherein the propellant is present in an amount of between 4% and 80% by weight relative to the total weight of the composition.

24. The composition as claimed in claim 1, wherein the propellant is present in an amount of between 30% and 70% by weight relative to the total weight of the composition.

25. A cosmetic hair treatment process comprising:
applying the composition as claimed in claim 1 to hair.

26. A process comprising
applying the composition as claimed in claim 1 to hair in an amount effective for at least one of shaping or holding the hair.

27. An aerosol device comprising a container containing a cosmetic composition in the liquid phase and a propellant, wherein the cosmetic composition comprises, in a cosmetically acceptable medium:
(i) solid particles comprising at least 10% by weight of at least one silicate, and
(ii) at least one surfactant;
wherein said solid particles comprise less than 1% by weight of aluminum.

28. The aerosol device as claimed in claim 27, wherein the silicate is at least one of a sodium, magnesium or lithium silicate.

29. The aerosol device as claimed in claim 27, wherein the solid particles have a number-average primary size of between 2 nm and 1 μm.

30. The aerosol device as claimed in claim 27, wherein the solid particles comprising at least 10% by weight of at least one silicate have a number-average primary size of between 5 to 500 nm.

31. The aerosol device as claimed in claim 27, wherein the solid particles comprise at least 50% by weight of at least one silicate.

32. The aerosol device as claimed in claim 27, wherein the silicate—comprising particles are present in an amount of between 0.01% and 10% by weight relative to the total weight of the composition.

33. The aerosol device as claimed in claim 27, wherein the surfactant is present in an amount of between 0.1% and 10% by weight relative to the total weight of the composition.

34. The aerosol device as claimed in claim 27, wherein the surfactant is an amphoteric or nonionic surfactant.

35. The aerosol device as claimed in claim 27, wherein the propellant is at least one selected from the group consisting of dimethyl ether, a C₃ to C₅ alkane, n-butane, propane, isobutene, halohydrocarbon, 1,1-difluoroethane, a mixture of dimethyl ether and a C₃₋₅ alkane, and a mixture of 1,1-difluoroethane and at least one of dimethyl ether or a C₃₋₅ alkane.

36. The aerosol device as claimed in claim 27, wherein the propellant is at least one of dimethyl ether, a C₃ to C₅ alkane, or a mixture thereof.

37. The aerosol device as claimed in claim 27, wherein the propellant is present in an amount between 2% and 90% by weight relative to the total weight of the composition.

38. The aerosol device as claimed in claim 27, wherein the composition, further comprises one or more cosmetic additives selected from the group consisting of an adhesive, a reducing agent, a thiol; a fatty substance; a thickener; a softener; an antifoam; a screening agent; an antiperspirant; an acidifying agent; a basifying agent; a dye; a pigment; a fragrance; a preserving agent; an anionic, cationic, nonionic or amphoteric, fixing or nonfixing polymer; volatile or nonvolatile, modified or unmodified, soluble or insoluble silicone; a plant, animal, mineral or synthetic oil, fragrance, protein, vitamin or glycerol; and mixtures thereof.

39. The aerosol device as claimed in claim 27, wherein the solid particles comprising at least 10% by weight of at least one silicate have a number-average primary size of between 10 and 250 nm.

40. The aerosol device as claimed in claim 27, wherein the solid particles comprise at least 70% by weight of at least one silicate.

41. The aerosol device as claimed in claim 24, wherein the solid particles comprise at least 90% by weight of at least one silicate.

42. The aerosol device as claimed in claim 27, wherein the silicate-comprising particles are present in an amount of between 0.5% and 3% by weight relative to the total weight of the composition.

43. The aerosol device as claimed in claim 27, wherein the surfactant is present in an amount of between 0.5% and 8% by weight relative to the total weight of the composition.

44. The aerosol device as claimed in claim 27, wherein the surfactant is present in an amount of between 1% and 5% by weight relative to the total weight of the composition.

45. The aerosol device as claimed in claim 27, wherein the propellant is present in an amount of between 4% and 80% by weight relative to the total weight of the composition.

46. The aerosol device as claimed in claim 27, wherein the propellant is present in an amount of between 30% and 70% by weight relative to the total weight of the composition.
